(19) United States
(12) Patent Application Publication
(10) Pub. No.: US 2023/0172734 A1
(43) Pub. Date: Jun. 8, 2023

(54) REMOVABLE STENT

(71) Applicant: Subroto PAUL, New York, NY (US)

(72) Inventors: Subroto Paul, New York, NY (US); Harish Seethamraju, Ridgewood, NJ (US)

(21) Appl. No.: 17/923,968

(22) PCT Filed: May 11, 2021
(86) PCT No.: PCT/US2021/031692

§ 371 Date: Nov. 8, 2022

Related U.S. Application Data
(60) Provisional application No. 63/022,955, filed on May 11, 2020.

(51) Int. Cl. A61F 2/915

(52) U.S. Cl. CPC A61F 2/915; A61F 2210/0023

(57) ABSTRACT

A retrievable medical stent is disclosed herein. The stent may include a substantially cylindrical configuration defining a longitudinal axis, an open proximal end, an open distal end, a stent lumen extending therebetween, and a contracting member circumscribing at least one of the open proximal end or the open distal end.

### CROSS-REFERENCE TO RELATED APPLICATIONS

This application claims priority to U.S. Provisional Pat. Application Ser. No. 63/022,955 filed May 11, 2020, the contents of which are incorporated herein by reference in their entireties.

### TECHNICAL FIELD

The present disclosure is generally directed toward medical stents, and more specifically, toward medical stents optimized for retrievability.

### BACKGROUND

A wide variety of medical stents may be designed for temporary placement in various body lumens or vessels for the treatment of disease or trauma, such as in the esophagus, gastrointestinal tract, trachea, arteries, aorta, etc., for example. Although designed for temporary deployment, a medical stent may reside at the site of placement for significant amounts of time, and as such, the natural response of the body may result in tissue ingrowth into the interstices of a stent scaffold or around the edges or terminal openings of the tubular portions of a stent. In some cases, the stent may be designed to oppose tendencies to migrate away from the site of placement, and with design elements that are antagonistic to retrieval following such migration, the problem of stent removal complicated by tissue ingrowth may be amplified.

Removable stents may incorporate one or more retrieval sutures at or near the open ends of, or extending circumferentially around, the stent. A retrieval device (forceps, hooks, etc.) may be used to grasp or hook the retrieval suture. However, such sutures may break during the removal process because of limitations of the strength of the suture material and the requisite thin diameter of the suture. Such breakage of retrieval sutures may exacerbate the removal process by eliminating contact points for grasping the stent, interrupting mechanical actuation of stent collapse afforded by the suture, or causing surgical complications due to broken pieces of the suture migrating away from site of the stent. Therefore, current design elements of so-called retrievable medical stents can be problematic and cause further traumatization and/or local tissue damage during removal. While stents have been designed to impede tissue ingrowth (e.g. using various coatings over the stent scaffold material) and stent construction materials have improved in strength, there remains a significant need for the improvement in stent design and construction for easier retrieval and reduced trauma at the stent deployment site.

### SUMMARY

The present disclosure may comprise one or more of the following features and combinations thereof.

This disclosure provides designs of, materials of, manufacturing methods for, and methods of use for, medical devices.

Stent configurations described herein may include the use of shaped memory materials to allow for the substantial contraction in size of the terminal ends of a deployed medical stent to afford easier retrieval by the technician, minimize local tissue trauma caused by the removal or retrieval process, and eliminate the need for retrieval sutures, hooks, loops and the like. A typical example stent of the present disclosure may include a substantially cylindrical configuration defining a longitudinal axis, an open proximal end, an open distal end, a stent lumen extending therebetween, and a contracting member circumscribing at least one of the open proximal end or open distal end. The contracting member may have a first shape memory material.

In some embodiments, the first shape memory material may include a first activation energy for a martensitic phase to an austenitic phase transition, and the martensitic phase to an austenitic phase transition may decrease a diameter of the at least one of the open proximal end or the open distal end. Additionally, in some embodiments, the substantially cylindrical configuration may include a second shape memory material, and the second shape memory material may have a second activation energy for a martensitic phase to an austenitic phase transition that is substantially different from the first activation energy.

In some embodiments, a first activation energy may be applied to the first shape memory material to cause a first conformational change in the at least one of the open proximal end or the open distal end, and a second activation energy may be applied to the second shape memory material to cause a second conformational change in the substantially cylindrical configuration. Additionally, in some embodiments, the first shape memory material may contract a diameter of the at least one of the open proximal end or open distal end to one of the following: less than about 5%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, and less than about 95% a diameter of a lumen wall in which the stent is positioned.

In some embodiments, the second shape memory material may contract a diameter of the substantially cylindrical conformation to less than one of the following: less than about 5%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, less than about 95% a diameter of a lumen wall in which the stent is positioned. Additionally, in some embodiments, the first shape memory material may include a first nitinol alloy. In some embodiments still, the second shape memory material may be a second nitinol alloy different from the first nitinol alloy.

These and other features of the present disclosure will become more apparent from the following description of the illustrative embodiments.

### BRIEF DESCRIPTION OF THE FIGURES

The invention described herein is illustrated by way of example and not by way of limitation in the accompanying figures. For simplicity and clarity of illustration, elements illustrated in the figures are not necessarily drawn to scale. For example, the dimensions of some elements may be exaggerated relative to other elements for clarity. Further, where considered appropriate, reference labels have been repeated among the figures to indicate corresponding or analogous elements.

FIG. 1 shows a side view of an example stent;

FIG. 2(a) shows one cross-sectional view of the stent shown in FIG. 1;

FIG. 2(b) shows another cross-sectional view of the stent shown in FIG. 1;

FIG. 2(c) shows yet another cross-sectional view of the stent shown in FIG. 1;

FIG. 3 shows a stent compressed around a stent delivery catheter shaft using a crocheted suture cord;

FIG. 4 shows a partially free-standing stent;

FIG. 5 shows an example stent with retrieval loops and without a contracting member;

FIG. 6 shows an uncoated example stent without a contracting member;

FIG. 7(a) shows a nitinol wire spring;

FIG. 7(b) shows the wire spring of FIG. 7(a) expanded such that the wire spring may be intertwined in a stent open end;

FIG. 8(a) shows an expanded nitinol spring wrapped around a stent;

FIG. 8(b) shows the spring of FIG. 8(a) intertwined with a stent open end;

FIG. 9(a) shows an example uncoated stent with a contraction member in a deployed condition;

FIG. 9(b)shows with the contraction member of the stent of FIG. 9(a) compressed in a Blockman crimper;

FIG. 10(a) shows an example stent with a contracting member in a deployed configuration;

FIG. 10(b) shows another view of the stent of FIG. 10(a);

FIG. 11(a) shows an example stent with a contraction member in a retrieval configuration;

FIG. 11(b) shows another view of the stent of FIG. 11(a);

FIG. 11(c) shows another view of the stent of FIG. 11(a);

FIG. 12(a) shows a coated example stent without a contracting member;

FIG. 12(b) shows another view of the stent of FIG. 12(a);

FIG. 13 shows a coated example stent with a contracting member in a deployed configuration;

FIG. 14(a) shows an example coated stent with a contracting member in a deployed condition;

FIG. 14(b)shows the stent of FIG. 14(a)with the contraction member compressed in a Blockman crimper; and

FIG. 15 shows an example coated stent with a contraction member in the retrieval configuration.

### DETAILED DESCRIPTION

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described. Additionally, it should be appreciated that items included in a list in the form of “at least one A, B, and C” can mean (A); (B); (C); (A and B); (A and C); (B and C); or (A, B, and C). Similarly, items listed in the form of “at least one of A, B, or C” can mean (A); (B); (C); (A and B); (A and C); (B and C); or (A, B, and C).

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative figures. Additionally, the inclusion of a structural or method feature in a particular figure is not meant to imply that such feature is required in all embodiments and, in some embodiments, may not be included or may be combined with other features.

A number of features described below may be illustrated in the drawings in phantom. Depiction of certain features in phantom is intended to convey that those features may be hidden or present in one or more embodiments, while not necessarily present in other embodiments. Additionally, in the one or more embodiments in which those features may be present, illustration of the features in phantom is intended to convey that the features may have location(s) and/or position(s) different from the locations(s) and/or position(s) shown.

Stents are used to support body lumens or vessels (e.g., lumens or vessels of the esophagus, gastrointestinal tract, trachea, arteries, aorta, etc.,) for treatment of disease or trauma. When deployed, they open up narrow strictures (e.g., such as areas covered by tumor or other tissue or areas that provide support to damaged tissue). Specifically, stents can be applied for support in collapsible airways or gastrointestinal segments. Thus, airway and gastrointestinal stents may be important for the treatment of tumors or other tissue growth blocking the aerodigestive system, radiation, trauma, or surgery related strictures, or to cover segments of the aerodigestive that collapse. Stents may also be used after surgery to support aerodigestive tissue that has inadequately healed. Stents may be frequently employed to treat both benign and malignant conditions in the aerodigestive system.

Stents are not without certain limitations. Tissue in the aerodigestive system can react to the presence of a foreign body and form excess reactive tissue overgrowth or granulation tissue around the stent. Although granulation can help secure the stent in position, more often the tissue overgrowth forms at the proximal and distal edges of the deployed stent, resulting in the difficulty of its removal.

Some stent designs include retrieval sutures or loops attached to the edges of the distal or proximal end of the stent to be used to pull the stent out of the placement and/or deployment position. If such a retrieval loop is not present, the stent must be grasped with a device and pulled out. However, the granulation tissue in-growth at the ends of the stent may cover the retrieval loop or the stent edges such that the retrieval loop or stent wall cannot be grasped, thereby exacerbating the retrieval process.

Other methods (for example, when granulation impedes removal) to retrieve the stent from its deployment location may include debulking the granulation tissue using a laser, cryoprobe, or mechanical grasper, or displacing it with a balloon placed between the stent and the wall of the lumen. Such techniques may add significant time to the surgical procedure and expose the patient to significant and unwanted risks, including damage to surrounding normal tissue from the accessory modalities needed to remove the granulation tissue, as well as stent fracture such that the stent is broken and comes out in pieces, for example. Stent fracture can be particularly worrisome as some parts of the stent may become imbedded in tissue away from the surgical site, complicating removal and leading to more granulation tissue. Furthermore, failure to completely remove stent fragments can be particularly hazardous if the pieces migrate into major vascular structures, or if the placement of other required stents, or additional surgical interventions, are required. Additionally, such procedures may require extended anesthetic time required to remove the stent, providing a further challenge to severely or critically ill patients.

Covered nitinol stents with a silicone polymer coating may be utilized to minimize the tissue in-growth resulting from stent deployment. However, those coated stents may be prone to granulation tissue forming at the proximal and distal edges of stent, preventing its retrieval by use of the retrieval loop or stent edge. Therefore, a need exists for a stent that is optimized for efficient and safe removal from a patient, including, for example, removal from the lumen of the aerodigestive tract.

The present disclosure provides a unique surgical stent with a modified proximal or distal end that contracts into a diameter smaller than the deployed diameter upon the application of an external energy source such as thermal or electrical energy, for example. The proximal or distal end of the stent then moves away from the wall of the lumen to facilitate grasping the edge of the stent or retrieval from the deployment location via the retrieval suture/loop. The following description should be read with reference to the drawings in which similar elements in different drawings are numbered the same. The drawings, which are not necessarily to scale, depict illustrative embodiments and are not intended to limit the scope of the disclosure.

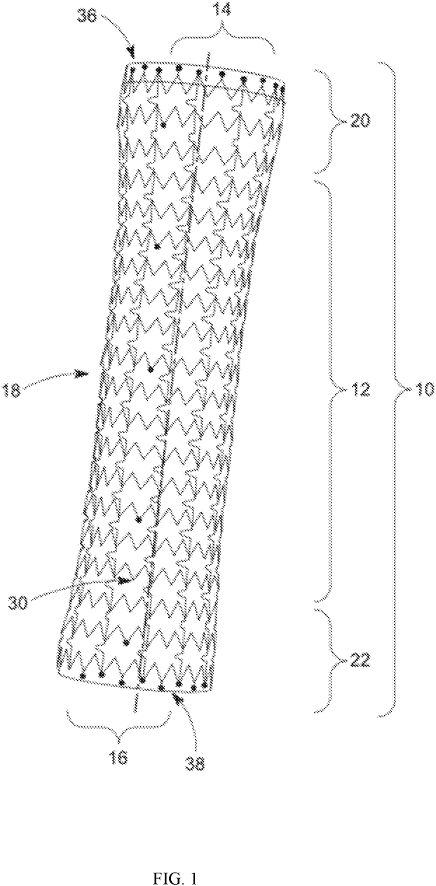

A typical example stent is comprised of a substantially cylindrical configuration with a longitudinal axis, the cylindrical configuration defining an inner surface, an outer surface, an open proximal end, and an open distal end. FIG. 1 shows an example stent 10. The cylindrical configuration can be comprised of a tubular framework 12 having an open first end 14, which may extend to the first end of the stent 10, and an open second end 16, which may extend to the second end of the stent 10, and a lumen extending therethrough. When positioned in a body lumen or vessel (e.g., in the esophagus, gastrointestinal tract, trachea, arteries, aorta, etc.), the open first end 14 of the stent may be considered the proximal end at which bodily fluids, food, etc. enter the stent 10, and the open second end 16 may be considered the distal end at which bodily fluids, food, etc. exit the stent.

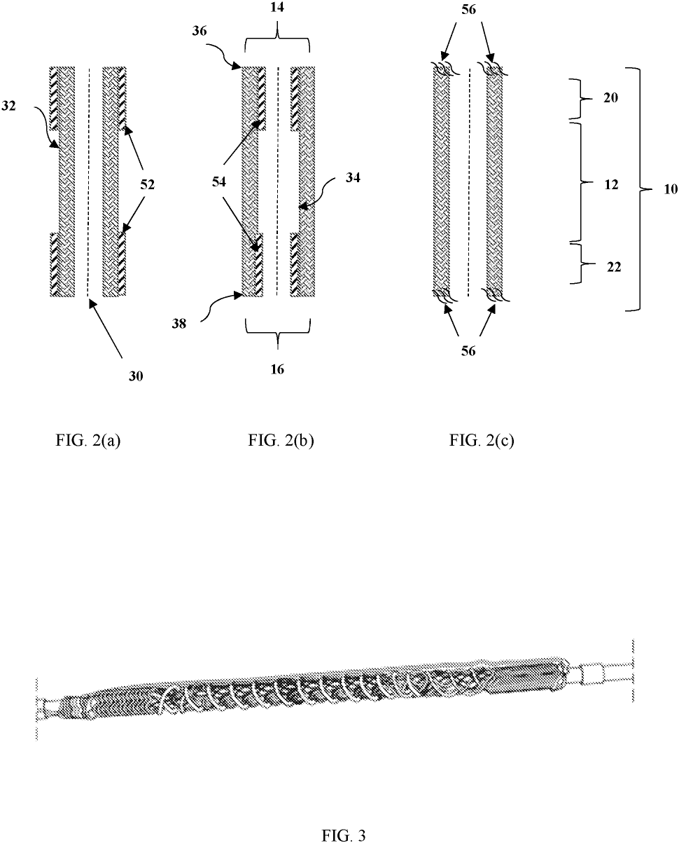

The tubular framework 12 may be considered the main support network of the stent. It should be appreciated that in some embodiments, such support networks may be, for example, formed of one or more of a certain stent filament material, or a plurality of stent filament materials. Additionally, in some embodiments, stent filaments 18 may extend continuously along the stent 10 from the open first end 14 to the second open end 16, as indicated by the longitudinal axis 30 (FIG. 2). In some embodiments still, the tubular framework of the stent 10 may be a monolithic structure formed from a continuous cylindrical tubular member extending from the open first end 14 to the second open end 16.

In certain embodiments of the present disclosure, the tubular framework 12 of the stent 10 may be associated with, or be attained by performing, an external mechanical expansion operation (for example, by way of a balloon mechanism, or a network of actuating members comprising the tubular scaffold). In other embodiments, however, the stent 10 may be a self-expanding stent. A tubular framework 12 that is self-expanding may include one or more woven stent filaments 18 of a same or different type of filament having filamentous wires that are braided, knitted, woven or otherwise interlaced. In some embodiments, the tubular framework 12 may be a monolithic or mesh structure. In other embodiments, the stent 10 or its components including the tubular framework 12 may be constructed from polymeric materials, as provided herein. In other embodiments still, the stent 10 or its components including the tubular framework 12 may be constructed of an alloy such as a nitinol alloy, etc., shape memory materials, a polymeric material, or a combination of metal alloy (such as a nitinol alloy, etc.) and at least one other polymeric material.

As shown in the longitudinal cross-section of the stent of the present disclosure (FIGS. 2a-2c), the stent 10 is particularly characterized by a contracting region 20 located at about the open first end, or a contracting region 22 located at about the open second end. The first contracting region 20 or second contracting region 22 may include a contracting scaffold that has the tubular framework 12 connected to a contracting member. The contracting member can be an outer surface contracting member 52 located circumferentially and generally about the outer surface 32 of the tubular framework 12 (e.g., FIG. 2(a)), an inner surface contracting member 54 located circumferentially and generally about the interior surface 34 of the tubular framework 12 (e.g., FIG. 2(b)), or a contracting crown member 56 located circumferentially and generally about the edge of the contracting region open first end 36 or the edge of the contracting region open second end 38 (e.g., FIG. 2(c)).

In certain embodiments, a contracting member 52, 54, or 56 may include one or more filamentous wires of a same or different type of material or alloy that exhibit contractile properties as described herein. The one or more filamentous wires comprising the contracting member 52, 54, or 56 may be braided, knitted, woven, coiled or otherwise interlaced, or may be a mesh structure, and exhibit contractile properties as described herein. In certain embodiments, the contracting crown member 56 may be a coil of one wire or a plurality of wires exhibiting contractile properties (which may be woven, braided, knitted, welded, glued, connected with resins etc.,) that are arranged circumferentially and generally near or about the edge of open first end 36 or the edge of the open second end 38, thereby forming a crown-like architecture. In other embodiments, a wire or wires including the plurality of wires of the contracting member 52, 54, or 56 may have a diameter of between about 0.006 inches to about 0.008 inches, between about 0.008 inches to about 0.010 inches, between about 0.010 inches to about 0.012 inches, between about 0.012 inches to about 0.014 inches, between about 0.014 inches to about 0.016 inches, between about 0.016 inches to about 0.018 inches, between about 0.018 inches to about 0.020 inches, and combinations thereof. A preferred diameter of wire or wires including the plurality of wires of the contracting member 52, 54, or 56 depends upon the force needed to overcome the hoop stress of the stent (e.g., the force required to compress a stent to a smaller circumferential diameter). It should be appreciated that the force depends upon the stent design and whether the stent is a covered stent (e.g., whether the stent has silicone or any other polymer coating along the body of the stent) to overcome the resistance offered by the granulation tissue. Diameter of a shaped memory alloy wire may also affect the manufacturing process for the stent as described herein. For example, nitinol wires that are too thick (i.e., greater than 0.040”) may provide sufficient contractile force but may be too difficult to manipulate for manufacturing. Thinner wires may provide insufficient contractile force for a desired placement, but may be easier to use in a manufacturing process. In this latter case, a thinner wire may be applied to a stent to more than one circumferential length to increase the contractile force. In certain embodiments of the present disclosure, the length of the shape memory wire or plurality of wires is sufficient to circumscribe a stent at least once. In other embodiments, the length of the shape memory wire or plurality of wires is sufficient to circumscribe a stent more than once.

In other embodiments still, the contracting member 52, 54 or 56 can be a monolithic structure, such as a band, ribbon, sheath, sleeve, etc. of shape memory material that includes a nitinol alloy circumscribing the interior or exterior surfaces of the tubular framework 12 located about first open end 14 or second open end 16 or edges 36 or 38 thereof, for example. The monolithic band, ribbon, sheath, sleeve, etc. of material can be an appropriate thickness and width to impart desired contractile properties (e.g., contractile force sufficient to separate from the lumen body at the deployment site). In certain embodiments, the monolithic band, ribbon, sheath, sleeve, etc. surface width may be less than 0.1 millimeters (mm), between about 0.1 mm and about 0.2 mm, between about 0.2 mm and about 0.3 mm, between about 0.3 mm and about 0.4 mm, between about 0.4 mm and about 0.5 mm, between about 0.5 mm and about 0.6 mm, between about 0.6 mm and about 0.7 mm, between about 0.7 mm and about 0.8 mm, between about 0.8 mm and about 0.9 mm, between about 0.9 mm and about 1.0 mm, or greater than about 1 mm. In other embodiments, the band or ribbon width can be less than 1.0 mm, between about 1.0 mm and about 5.0 mm, between about 5.0 mm and about 10.0 mm, between about 10.0 mm and about 15.0 mm, between about 15.0 mm and about 20.0 mm, between about 20.0 mm and about 25.0 mm, or greater than about 25.0 mm.

The contracting region at the open first open end 20 or open second end 22 may be in an expanded conformation or a contracted conformation. In the expanded conformation, the open first end 14 or the open second end 16 is expanded after deployment to a position in a lumen, and exhibits a circumference approximately the same as or larger than the lumen that is to impart a radial force on the lumen wall. In the contracted conformation, the contracting region exhibits a circumference than about 5%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, or less than about 95% of a circumference of a lumen in which the stent is deployed.

It can be appreciated that the amount of contractile force generated by any of the contracting members 52, 54, or 56 can be proportional to the contracting member material utilized, such as a shape memory alloy (e.g., nitinol), in the construction of the contracting region 20 or 22. Generally, the greater the amount of shape memory material used in the construction of any of the contracting members 52, 54, or 56, the greater the contractile force generated upon activation (described more fully herein). Certain tissues where the stent 10 is deployed may be differentially prone to localized damage (for example tissue tearing) when the deployed stent is contracted for retrieval because of natural tissue in-growth into or on the stent. For example, lumen regions with greater tissue thickness or inherent strength may be able to accommodate more shape memory material in the structure of any of the contracting members 52, 54, or 56. Conversely, lumen regions with smaller tissue thickness may only be able to accommodate smaller amounts of shape memory material in the structure of any of the contracting members 52, 54, or 56. At the same time, it can be appreciated that the amount of shape memory material in any of the contracting members 52, 54, or 56 may be practically limited by the specific medical application and location of anatomical deployment. For example, lumen regions with large diameters may be able to accommodate more shape memory material in any of the contracting members 52, 54, or 56. Conversely, smaller lumen diameters may only be able to accommodate smaller amounts of shape memory material in any of the contracting members 52, 54, or 56. Therefore, to limit localized trauma upon contraction and retrieval of the stent, it can be appreciated that shape memory material in any of the contracting members 52, 54, or 56 can be configured and optimized to modulate the amount of contractile force as appropriate for the tissue in the deployment location. In certain embodiments where the contracting member 52, 54, or 56 includes of a shape memory material such as nitinol, wire diameter, the number or pitch of the coils will determine the amount of force imparted during contraction. [see Examples].

Materials useful for the construction and function of the disclosure provided herein describe the use of shape memory polymers and alloys. Nitinol, a titanium and nickel alloy providing shape memory associated with thermal activation, has been widely adapted to use in medical devices such as catheters or stents having a self-expanding function. In preferred embodiments of the present disclosure, an alloy of nitinol includes the contracting member 52, 54 or 56. The thermal activation characteristics of nitinol and nitinol alloys allows such devices to be constructed of the alloy to a desired shape while above a characterized threshold temperature (the austenitic state) and deformed to an alternative structure below a characteristic threshold temperature for deployment (the martensitic state). When subjected to thermal energy above the threshold temperature in the martensite state, the deformed device re-acquires its original austenitic state shape. The two interchangeable shapes are possible because of the two distinct microcrystalline structures that are interchangeable with a small variation in temperature. The temperature at which a medical device assumes its first configuration may be varied within a wide temperature range by manufacturing nitinol alloys that differ in the ratios of titanium and nickel or by forming nitinol alloy composites.

By forming the body or portions thereof of a medical device with a nitinol alloy, such as a nitinol alloy mesh or wire, a transition between the martensitic state and the austenitic state can be achieved by temperature transitions above and below a transition temperature or transition temperature range. Commonly, these controlled temperature transitions are employed to soften a device for easier deployment to an anatomical site.

In certain embodiments of the present disclosure, it can be understood that medical stents may be made with or modified and improved with a nitinol alloy including one of the contracting members 52, 54, or 56 of the present disclosure. Such manufacturing or modifications can be made to those medical stents by connecting one of the contracting members 52, 54, or 56 as described herein using techniques known in the art including, for example, weaving, braiding, knitting, welding, gluing, bonding, etc.

In other certain embodiments of the present disclosure, certain sections of the retrievable stent 10 can be designed and manufactured using at least two forms of nitinol alloy, one form in each section, wherein two conformational shape changes in the retrievable stent 10 can be possible as attributable to the different thermal activations of the alloys. For example, it can be understood that the tubular framework 12 of the retrievable stent 10 may be constructed of a nitinol alloy having a thermal activation temperature at about or below average human physiological temperature, and each of the contracting members 52, 54 or 56 may be constructed of a nitinol alloy having a thermal activation energy substantially higher, for example greater than 10° C., greater than 20° C., greater than 30° C., greater than 40° C., greater than 50° C., greater than 60° C., greater than 70° C., greater than 80° C., greater than 90° C., greater than 100° C., or higher, than average human physiological temperature or the thermal activation temperature of the nitinol alloy used in the construction of the tubular framework 12. In this example it can be understood that the retrievable stent 10 can be deployed when both nitinol alloys are in the martensitic state, wherein the tubular framework 12 can be in a structurally deformed, collapsed martensite state, and any of the contracting members 52, 54 or 56 can be in a structurally deformed expanded martensitic state. Upon deployment to the anatomical site, the nitinol alloy including the tubular framework and characterized by the thermal activation temperature at around human physiological temperature would acquire its austenitic phase and expand to the shape of the lumen, and the nitinol alloy of the contracting member 52, 54 or 56 would retain the martensitic phase shape. When retrieval of the retrievable stent 10 is desired, thermal activation of the contracting 52, 54 or 56 member would cause a material phase shift from the martensitic state to the austenitic state and the contracting member 52, 54 or 56 would contract the overall diameter of the open end 20 or open end 22 of the stent.

In other certain embodiments of the present disclosure, the contracting member 52, 54, or 56 or the stent framework 12 can include other or additional shape memory alloys or other polymeric materials, etc., exhibiting contractile properties when exposed to an external energy source. Other embodiments of the present disclosure include the use of other shaped memory materials that may be used as a contraction mechanism in, for example, about the open ends 20 or 22 of a deployed medical stent. Such materials may include natural polymers, metal alloys, plastics, piezoelectric materials, etc. Representative shape-memory polymers can include polyurethanes, polyurethanes with ionic or mesogenic components made by prepolymer method. Other block copolymers also show the shape-memory effect, such as, block copolymer of polyethylene terephthalate (PET) and polyethyleneoxide (PEO), block copolymers containing polystyrene and poly(1,4-butadiene), and an ABA triblock copolymer made from poly(2-methyl-2-oxazoline) and polytetrahydrofuran. A linear, amorphous polynorbornene (Norsorex, developed by CdF Chemie/Nippon Zeon) or organic-inorganic hybrid polymers consisting of polynorbornene units that are partially substituted by polyhedral oligosilsesquioxane (POSS) also have shape-memory effect, as well as chemically crosslinked shaped memory polymers such as crosslinked polyurethane, PEO-PET block copolymers can be crosslinked by using maleic anhydride, glycerin or dimethyl 5-isophthalates as a crosslinking agent, AA/MAA copolymers, MAA/N-vinyl-2-pyrrolidone, PMMA/N-vinyl-2-pyrrolidone, polyaryl etherketones such as polyether ether ketone, light induced shaped memory materials induced by light, for example cinnamylidene acetic acid, electrically induced shaped memory materials including carbon nanotubes, short carbon fibers (SCFs), carbon black, or metallic Ni powder, magnetic shaped memory alloys including Nickel-Manganese-Gallium (Ni-Mn-Ga) alloys, Iron-Palladium (Fe-Pd) alloys, Nickel-Iron-Gallium (Ni-Fe-Ga) alloys, and several derivates of the basic Ni-Mn-Ga alloy which further contain Iron (Fe), Cobalt (Co) or Copper (Cu) and composites thereof; natural polymers including zein, casein, gelatin, gluten, serum albumin, collagen, polysaccharides, polyhyaluronic acid, poly(3-hydroxyalkanoate)s, alginate, dextran, cellulose, collagen, synthetic polymers, chemical derivatives of collagen, chemical derivatives of cellulose, polyphosphazenes, poly(vinylalcohols), polyamides, polyacrylates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyesters, degradable polymers, polyester amides, polyanhydrides, polycarbonates, polyorthoesters, polylactides, polyglycolides, polysiloxanes, polyurethanes, cellulose derivatives, polynorbomene, polycaprolactone, polyenes, nylons, polycyclooctene (PCO), blends of PCO and styrene-butadiene rubber, polyvinyl acetate/polyvinylidinefluoride (PVAc/PVDF), blends of PVAc/PVDF/polymethylmethacrylate (PMMA), polyurethanes, styrene-butadiene copolymers, polyethylene, trans-isoprene, blends of polycaprolactone and n-butylacrylate; blends thereof; mixtures thereof; and other suitable materials.

While the shape memory polymers that are described above are activated by thermal energy, in certain embodiments of the present disclosure, the shape memory material can be activated by other forms of energy, e.g., light/photoinduction energy, electrical, ultrasound, plasma or microwave, etc., for example.

Medical stent materials compatible for manufacture with or modification by a shape memory contracting member 52, 54 or 56 as disclosed herein may include metal, metal alloy, polymer, a metal polymer composite, ceramics, combinations thereof, and the like, or other suitable materials. Examples of suitable polymers may include polytetrafluoroethylene, (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene, terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY®, alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material. However, this is not intended to be a limiting list of such materials, as the disclosure may be applied to other such materials appropriate for medical application.

In at least some embodiments, portions or all of stent may also be doped with, made of, or otherwise include a radiopaque material. Radiopaque materials are understood to be materials capable of producing a relatively bright image on a fluoroscopy screen or another imaging technique during a medical procedure. This relatively bright image aids the user of medical stent as disclosed herein in determining their locations. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of medical stent disclosed herein to achieve the same result.

In some embodiments, a degree of magnetic resonance imaging (MRI) compatibility is imparted into the medical stent of the present disclosure. For example, the medical stent 10, other components of the medical stent 10, or portions thereof, may be made of a material that does not substantially distort the image and create substantial artifacts (e.g., gaps in the image). The medical stent 10, other components of the medical stent 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. Having generally described this invention, a further understanding can be obtained by reference to certain specific examples, which are provided herein for purposes of illustration only, and are not intended to be limiting unless otherwise specified.

### EXAMPLES

To facilitate easy retrieval of an airway stent post-implant, a surgically induced contraction in the diameter of the stent edge circumference can cause a protrusion of the stent edge from the granulation tissue and into the airway lumen. The protruding (contracted) stent edge can enable the surgeon to grasp the stent with a retrieval tool and dislodge it from the granulation tissue, eliminating the current necessity for grasping the stent edge by trial and error because of the uncertainty in exactly finding the edge of the stent. As provided in this present disclosure, the contracted edge is more clearly identifiable within the lumen to initiate the stent retrieval process.

A shape memory material attached to an open end of a self-expanding medical stent provides an illustration. One example of a shape memory material is a nitinol alloy wire in the form of an actuator spring commercially marketed as Flexinol® shape memory alloy springs by Dynalloy Corporation (similar wires and springs are manufactured by NexMetal, Inc., Kelloog Nitinol, Inc. and others). To make the shape memory alloy springs, Flexinol® shape memory actuator wire is wound in the form of a spring and shape change temperature is programmed into the composition. At room temperature a Flexinol® actuator spring is in the martensite state i.e. is deformable at moderate loads and will not recover from the deformed state and will stay stretched. If in the deformed state the wire is cooled, no change occurs. But if the deformed Flexinol® actuator spring is heated to a transition temperature (“Tf”), it shrinks back to the original length in the shape of the spring. This process of bulk deformation or expansion and shrinkage can be repeated numerous numbers of times, and during shrinkage the Flexinol® actuator spring can exert a certain force. The transition temperature can be programmed in between any values between 45° C. to 100° C. In one application, the stretched-out Flexinol® actuator spring can be intertwined about the edge of a stent open end and the terminal ends of the Flexinol® actuator spring can be twist-tied or crimped to form a circumferential loop. The stent can then undergo its normal surgical preparation processes, i.e. crimping into a sheath on a catheter delivery system, packaging and sterilization.

At the time of implant, the self-expanding stent modified with the Flexinol® actuator spring can be deployed per its typical procedure. The Flexinol® actuator spring would be in the martensite state and will not affect the ability of the self-expanding stent to attain its full diameter in the deployed site in the airway. Post-deployment, within 3 months, granulation tissue generally develops around the stent edges, engulfing the entire exposed portion of the stent including the intertwined Flexinol® actuator spring. When it is time to retrieve the stent, the edge of the stent and Flexinol® actuator spring is exposed to the transition temperature using electrocautery or other means typically employed within the operation floor. When the Flexinol® actuator spring reaches transition temperature, it undergoes contraction and will pull in the edge of the stent into the lumen. The physician can then grab the stent open end edge and slowly remove the stent from the granulation tissue.

Use of a shaped memory alloy in a spring form enables several advantages. The springs can be made with a range of wire diameters, spring pitches and lengths. Therefore, a spring contraction force can be modulated for a specific tissue deployment by changing these wire parameters. For example, a thicker wire diameter for a specified wire length and spring pitch will result in a larger contraction force. Certain spring forms can be expanded, for example, up to ten times the original length, and the expanded wire can be intertwined within the edge of a stent without adding bulk to the stent or affect the crimp ability of the stent. When exposed to the Tf, the wire will contract back into the original length and number of spring pitches, and in doing so, will result in the contraction of the stent edge and a reduction in the circumference of the stent open end and subsequent protrusion of the open end into a lumen. If necessary, the cycle of deformation into a martensite phase without recovery, contraction at an applied transition temperature to its austenite phase, and cooling to martensite phase can be repeated number of times. If the deformation and contraction is needed only one time, a simple shape memory wire could also be fabricated and substitute the retrieval loops or threads commonly used inside commercial stents.

The attainable shrinkage can be exemplified for three possible forms of the shaped memory wire: (i) For a simple wire, the base wire used to fabricate shape memory alloy spring undergoes shrinkage but is limited to 8% its length. For instance, if a 100 mm wire of Flexinol® actuator wire is expanded under load to 108 mm, and then heated to Tf, it will shrink to 100 mm attaining (108-100)/100 = 8% shrinkage, and the expansion and shrinkage cycle can be repeated number of times; (ii) For a shape memory alloy spring, a 15 mm spring may be expanded to 100 mm length. When exposed to Tf, it will shrink back to ~ 15 mm, thus attaining (100-15)/15 = 560% shrinkage. This is possible because in this case the wire itself is not expanding but the spring is subjected to bulk deformation. In this case the expansion and shrinkage cycle can be repeated number of times; (iii) For a simple wire intended to utilize just one cycle of shape recovery, a simple shape memory wire can be made with a 15 mm long wire stretched under load to 100 mm and heated to Tf to contract back to 15 mm. In this case the wire itself is stretching and contracting but this cycle can be achieved only one time. The shaped memory wire described in (ii) and (iii) can be wound around the proximal or distal edge of the stent circumferentially by intertwining around the loops of the stent. The length of wire used is slightly more than circumference π x D, where D is a diameter of the stent. For example, if a stent outer diameter is 20 mm, the length of shaped memory wire described in (ii) and (iii) can be somewhere between approximately 63-126 mm (π x 20 = 63 mm). The length of shaped memory wire described in (i) provides other design options. For example, this wire form can be positioned along the length of the stent such that 8% contraction on a longer length can provide sufficient contraction of the edge of the stent.

The following examples illustrate the use of a Flexinol® shape memory alloy spring and its application to a medical stent and subsequent stent contraction when exposed to its transition temperature (Tf). A manual crimper manufactured by Blockman, Inc., was used to compress the stent without and with a shape memory wire intertwined circumferentially about an open stent end to determine the final stent diameter after exposure to Tf. The crimper can accommodate stents up to approximately 40 mm in longitudinal free length and a maximum diameter of approximately 30 mm.

### Example #1: Self-Expandable Bare Metal Stent

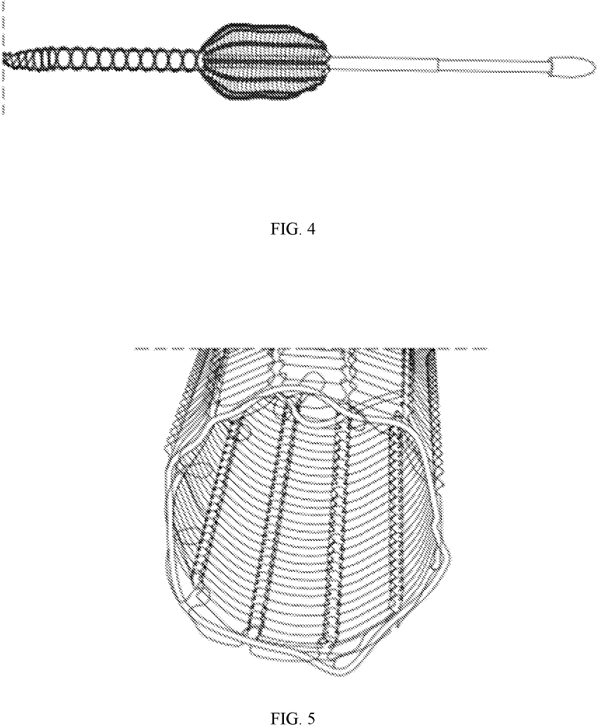

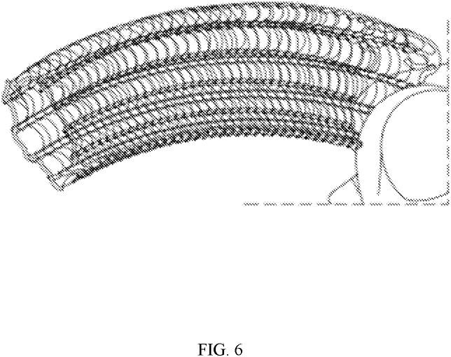

Stents were obtained from Boston Scientific Corporation that are 20 mm in diameter and 54 mm length free standing and compressed around a stent delivery catheter shaft using a crocheted suture cord (FIG. 3) to evaluate the diameter used for placement in a lumen. The stent was then configured into a “deployed” (expanded) state by partial unraveling of the suture cord (FIG. 4). The outer diameter of the crocheted suture with the compressed stent is approximately 18-19 Fr (6-6.3 mm; “Fr”; equivalent to about 0.33 mm/Fr), indicating about a 70% outer diameter compression to facilitate delivery into the deployment location. Free standing stent can be further compressed in the Blockman crimper with coolant aid until no further reduction in diameter can be attained, approximately 18 Fr (6 mm). As shown in FIG. 5, the stent has a heavier gauge silk thread (also located at the opposite end). FIG. 5 also shows retrieval loops under the strut housing the silk thread which would slightly protrude into a lumen right. Since this is an uncoated stent (i.e., no polymeric coating), the structure exhibits nominal longitudinal strength and appears flaccid when supported at one end (FIG. 6). Stents coated in a polymer are demonstrably stiffer.

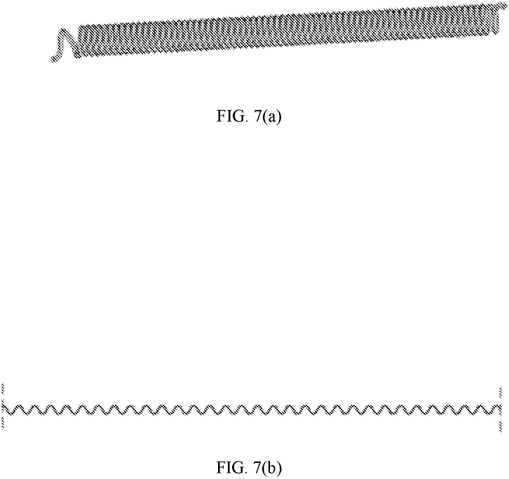

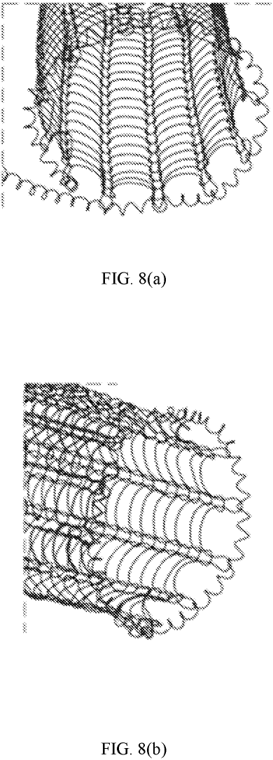

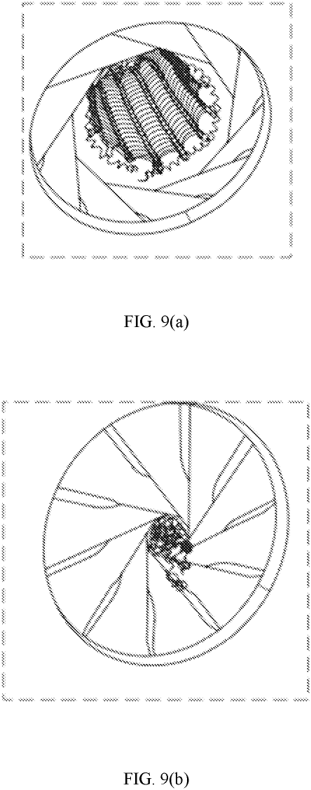

As described above, the desired contracted length of Flexinol® shaped memory spring, when positioned circumscribing the stent, corresponds to an approximately 18 Fr (6 mm) diameter. A Flexinol® wire spring including an 0.008” diameter wire, 0.054” spring diameter, and about 20 mm free length (π x 6.3 mm diameter = approximately 20 mm) was used FIG. 7(a)and the same wire shown expanded to intertwine a stent open end (FIG. 7(b). The length of the Flexinol® wire in the expanded condition is sufficiently long to circumscribe the expanded (deployed) stent (minimum spring expanded length is π x 20 mm (diameter) = 63 mm) and provide overlap for crimping the spring terminal ends. In FIGS. 8(a) and (b) is shown the expanded Flexinol® spring wrapped around a stent open end.

The stent in a deployed condition and with the expanded Flexinol® spring was then compressed in a Blockman crimper to a final diameter of 6 mm to represent the deployment diameter (FIG. 9(a)), as above. While compressing the stent, the Flexinol® spring is shown to protude from the crimper (FIG. 9(b)). This protrusion not considered a problematic to deployment as since a sheath can be placed over end of the crimped stent. The stent with expanded Flexinol® being compressed to a 6 mm crimped diameter confirms that with using conventional crimping procedures the desired reduction in diameter of the stent can be achieved to facilitate easy deployment into a lumen (for example, an airway). It is generally preferred that stents are inserted during implantation at lowest possible diameter to minimize trauma to the patients and improve access to the airway.

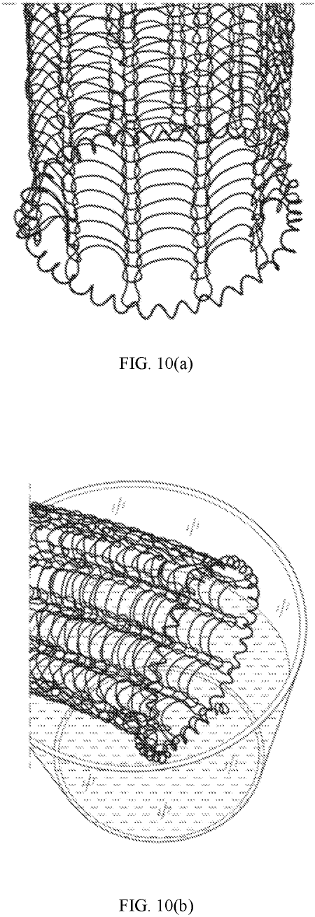

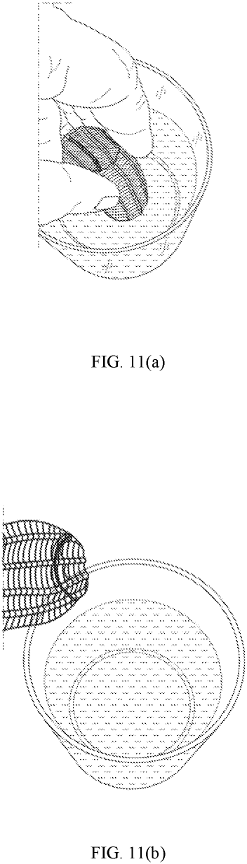

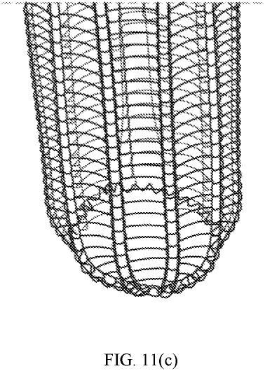

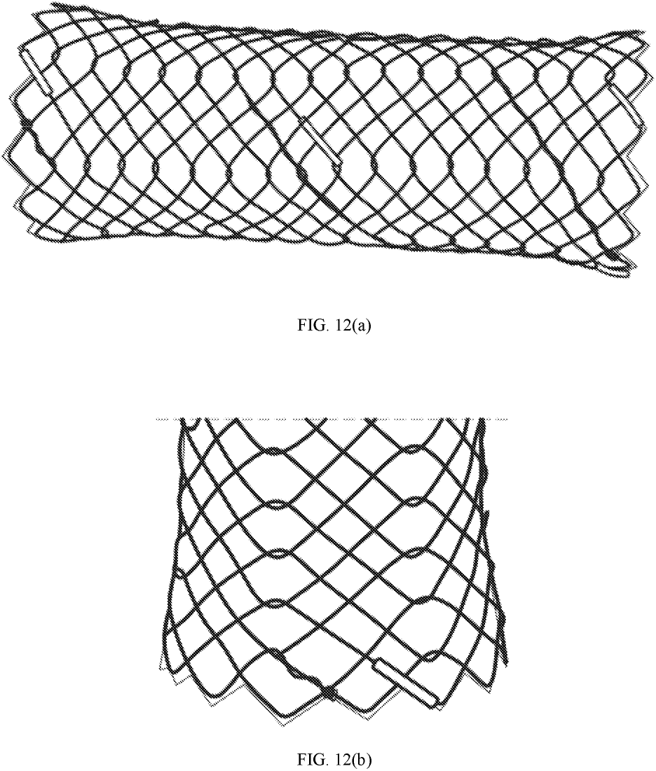

When the stent is removed from the crimper it regains the 20 mm outer diameter deployed diameter (FIGS. 10(a) and (b). When the stent is to be retrieved from the site of deployment, an external energy source at the Tf is contacted with the stent at the placement of the Flexinol® spring. Here, the concept is demonstrated with the application of hot water (< 100 C) to trigger the transition of the Flexinol® spring from its expanded state to its contracted state (converting from martensite to austenite phase (FIGS. 11(a)-(c)). The stent was compressed to approximately 15 mm with the addition of heat at the Tf due to the contraction of the Flexinol® spring. Importantly, the contraction region of the stent can be expanded, followed by additional application of the external energy source, for multiple cycling of the expanded/contracted configuration. This cycling can aid in the separation of the deployed stent from a lumen wall if the stent edges are impeded by tissue in-growth.

### Example 2: Self Expandable Covered Stent

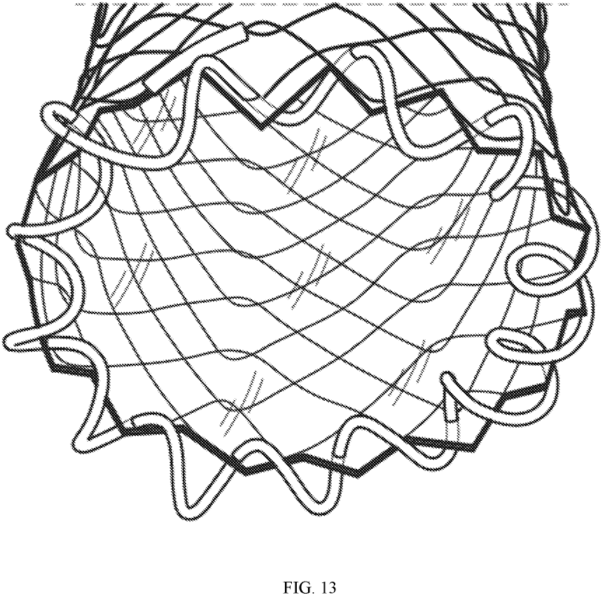

A polymer coated stent was modified with a Flexinol® spring at the stent open end to demonstrate the effectiveness of a shape memory spring wire to compress a substantially stiffer stent. The covered stent used here measured 40 mm free standing length and an outer diameter of 16 mm (FIGS. 12(a) and (b) ). The stent design with its added polymeric coating increases the hoop stress of the stent, thus requiring a greater compressive force by the Flexinol® spring to contract the stent from its deployed state to its retrieval state.

A shaped memory spring of 0.1” outer diameter, including a wire 0.015” thick is first expanded to approximately seven times the original length (using calculated requirements as described in Example 1). The extended spring was intertwined with the stent along the circumference of the covered stent as shown below at the stent open end (FIG. 13). The pitch of the expanded spring was chosen to match the hole pattern of the stent for easier intertwining, as shown below.

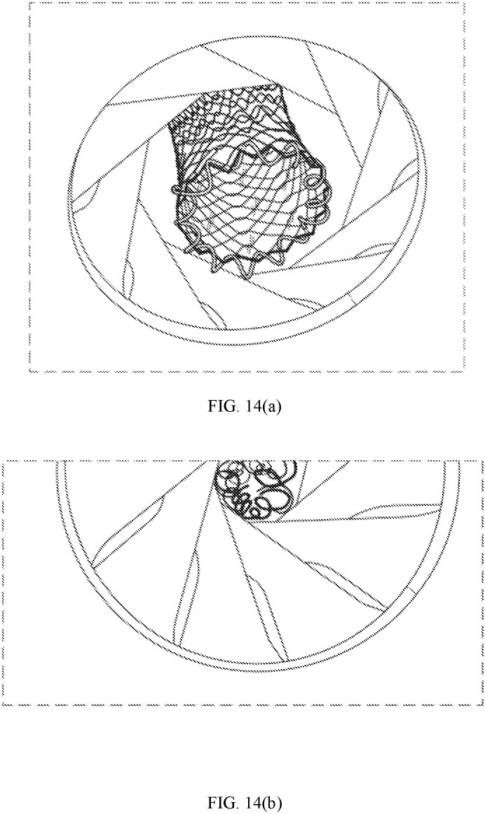

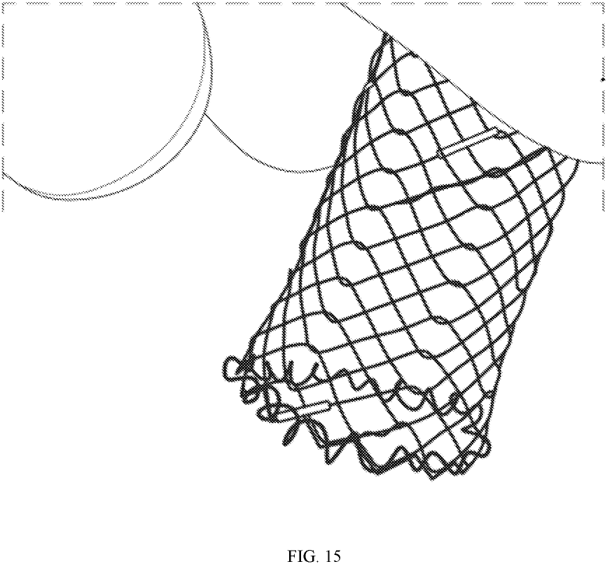

After intertwining the expanded spring, the edge of the stent was compressed under a cooling aided on the Blockman crimper to a diameter of approximately 8 mm to achieve 50% compression, emulating a condition for deployment to a lumen. The stent was removed from the crimper and both the stent and the intertwined spring recovered the original 16 mm outer diameter (FIGS. 14(a) and (b). The stent with the intertwined spring was exposed to hot water (Tf ~ 100C) wherein the shape memory spring contracted to a 10 mm outer diameter (FIG. 15).

For the following defined terms, these definitions shall be applied, unless a different definition is given in the claims or elsewhere in this specification.

The term “about” generally refers to a range of numbers that one of skill in the art would consider equivalent to the recited value (e.g. having the same function or result). In many instances, the term “about” may include numbers that are rounded to the nearest significant figure. All numeric values are herein assumed to be modified by the term “about”, whether or not explicitly indicated. The term “about” can also generally refer to a position or location, that one of skill in the art would consider equivalent to a recited position or location and having an equivalent function or result.

The recitation of numerical ranges by endpoints includes all numbers within that range. By way of non-limiting example, the range “1 to 5” would also include the values 1, 1.5, 2, 2.75, 3, 3.8, 4, 5.

As used in the specification and the appended claims, the singular forms “a”, “an”, and “the” include plural referents unless the context clearly dictates otherwise. As used in this specification and the appended claims, the term “or” is generally employed in its sense including “and/or” unless the context clearly indicates otherwise.

It is noted that references in the specification to “an embodiment”, “some embodiments”, “other embodiments”, etc., indicate that the embodiment described may include one or more particular features, structures or characteristics. However, such recitations do not necessarily mean that all embodiments include the particular features, structures or characteristics. Additionally, when particular features, structures or characteristics are described in connection with one embodiment, it should be understood that such particular features, structures or characteristics may also be used in connection with other embodiments whether or not explicitly set forth in the specification unless clearly stated to the contrary.

Nothing herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.” Furthermore, to the extent that the term “include,” “have,” or the like is used in the description or the claims, such term is intended to be inclusive in a manner similar to the term “comprise” as “comprise” is interpreted when employed as a transitional word in a claim.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents and alternatives falling within the spirit and scope of the disclosure.

What is claimed is:

1. A retrievable medical stent comprising:
a substantially cylindrical configuration defining a longitudinal axis, an open proximal end, an open distal end, a stent lumen extending therebetween, and a contracting member circumscribing at least one of the open proximal end or open distal end,
wherein the contracting member is comprised of a first shape memory material,
wherein the first shape memory material comprises a first activation energy for a martensitic phase to an austenitic phase transition, and
wherein when the medical stent is positioned in a body lumen or vessel, the martensitic phase to an austenitic phase transition decreases a diameter of the at least one of the open proximal end or the open distal end such that the at least one of the open proximal end or the open distal end moves away from a wall of the body lumen or vessel to facilitate grasping of the medical stent.

2. The stent of claim 1, wherein the substantially cylindrical configuration comprises a second shape memory material, wherein the second shape memory material comprises a second activation energy for a martensitic phase to an austenitic phase transition that is substantially different from the first activation energy.

3. The stent of claim 2, wherein a first activation energy applied to the first shape memory material causes a first conformational change in the at least one of the open proximal end or the open distal end, and a second activation energy applied to the second shape memory material causes a second conformational change in the substantially cylindrical configuration.

4. The stent of claim 1, wherein the first shape memory material contracts a diameter of the at least one of the open proximal end or open distal end to less than about 5%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, or less than about 95% a diameter of the wall when the stent is positioned in the body lumen or vessel.

5. The stent of claim 2, wherein the second shape memory material contracts a diameter of the substantially cylindrical configuration to less than about 5%, less than about 10%, less than about 15%, less than about 20%, less than about 25%, less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, or less than about 95% a diameter of the wall when the stent is positioned in the body lumen or vessel.

6. The stent of claim 2, wherein the first shape memory material is comprised of a first nitinol alloy.

7. The stent of claim 6, wherein the second shape memory material is comprised of a second nitinol alloy different than the first nitinol alloy.

8. In a medical stent comprising a substantially cylindrical configuration defining a longitudinal axis, an open proximal end, an open distal end, and a stent lumen extending therebetween, the improvement comprising a contracting member circumscribing at least one of the open proximal end or open distal end,
wherein the contracting member is comprised of a first shape memory material,
wherein the first shape memory material comprises a first activation energy for a martensitic phase to an austenitic phase transition, and
wherein when the medical stent is positioned in a body lumen or vessel, the martensitic phase to an austenitic phase transition decreases a diameter of the at least one of the open proximal end or the open distal end such that the at least one of the open proximal end or the open distal end moves away from a wall of the body lumen or vessel to facilitate grasping of the medical stent.

9. The stent of claim 8, wherein the substantially cylindrical configuration comprises a second shape memory material, wherein the second shape memory material comprises a second activation energy for a martensitic phase to an austenitic phase transition that is substantially different from the first activation energy.

10. The stent of claim 9, wherein a first activation energy applied to the first shape memory material causes a first conformational change in the at least one of the open proximal end or the open distal end, and a second activation energy applied to the second shape memory material causes a second conformational change in the substantially cylindrical configuration.
